(19) United States
(12) Patent Application Publication
(10) Pub. No.: US 2009/0185656 A1
(43) Pub. Date: Jul. 23, 2009

(54) CONE-BEAM CT HALF-CYCLE CLOSED HELICAL TRAJECTORY

(75) Inventor: Dominic J. Heuscher, Aurora, OH (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/302,091

(22) PCT Filed: May 9, 2007
(86) PCT No.: PCT/US07/68523

§ 371 Date: Nov. 24, 2008

Related U.S. Application Data
(60) Provisional application No. 60/803,158, filed on May 25, 2006.

(51) Int. Cl. A61B 6/00

(52) U.S. Cl. 378/11

(57) ABSTRACT

A tomographic apparatus (10) includes radiation source (20), at least one radiation sensitive detector (30), and a reconstruction system (40). The radiation source (20) sweeps along a z-axis (16) and returns to its initial position in coordination with about two revolutions of the radiation source (20) about an imaging region (32) with a frequency of about half a frequency of a revolution of the radiation source (20) about the imaging region (32). The at least one radiation sensitive detector (30) detects radiation emitted by the radiation source (20) that traverses a volume of interest (52) within the imaging region (32) and generates data indicative of the detected radiation. The reconstruction system (40) reconstructs the detected data to generate an image of a subject in the volume of interest (52).

The following relates to medical imaging systems. It finds particular application to computed tomography (CT) and, more particularly to a CT imaging approach for acquiring substantially complete sampling of a volume of interest (VOI) with efficient use of the radiation detectors.

With cone-beam CT, a complete data set or sampling of a VOI can be used to reconstruct the VOI. Conventional cone-beam CT scanning techniques that employ a circular (or axial) radiation source orbit (path, trajectory, etc.) around an imaging region fail to provide complete sampling. Instead, the resulting data set is not complete in that the sampling on either end to the sample VOI is incomplete. One approach for obtaining complete sampling with the circular orbit is to perform circle and/or line scans, and then combine the scans together.

In an alternative approach, a saddle radiation source orbit is used to achieve complete sampling of the VOI. Such a saddle orbit is described in “Investigation of a saddle trajectory for cardiac CT imaging in cone-beam geometry,” Pack et al., Phys. Med. Biol., vol. 49, No. 11 (2004) pp. 2317-2336. However, with the saddle orbit the width of the radiation detector in the z-direction is relatively larger than the detector width used with the circular orbit. This is due to a larger source trajectory. Thus, with the saddle orbit, complete sampling can be achieved at a cost of reduced detector efficiency. The increased detector size may lead to an overall increase in the cost of manufacturing the CT system since the radiation detectors account for a relatively large percentage of the total cost of such system.

In view of the above deficiencies with conventional techniques, there is an unresolved need for improved techniques for acquiring complete sampling of a VOI used to reconstruct the VOI while mitigating these as well as other deficiencies.

According to one aspect, a tomographic apparatus having radiation source, at least one radiation sensitive detector, and a reconstruction system is illustrated, The radiation source sweeps along a z-axis and returns to its initial position in coordination with about two revolutions of the radiation source about an imaging region with a frequency of about half a frequency of a revolution of the radiation source about the imaging region. The at least one radiation sensitive detector detects radiation emitted by the radiation source that traverses a volume of interest within the imaging region and generates data indicative of the detected radiation. The reconstruction system reconstructs the detected data to generate an image of a subject in the volume of interest.

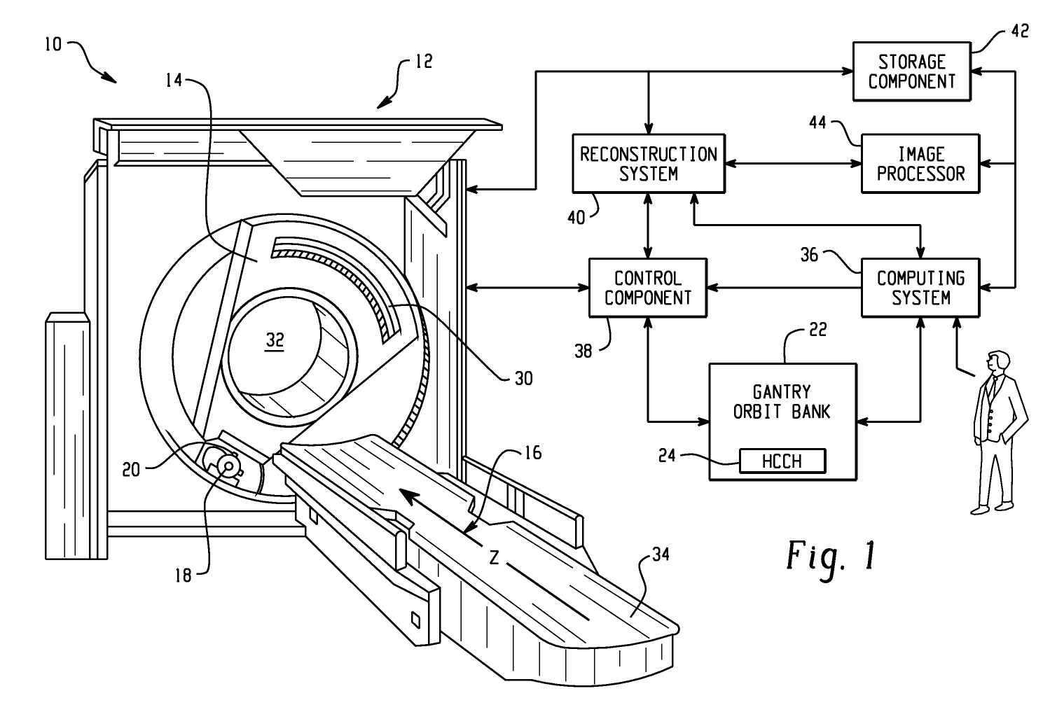

FIG. 1 illustrates an exemplary medical imaging system with a radiation source orbit that at least efficiently utilizes the width of the radiation detectors.

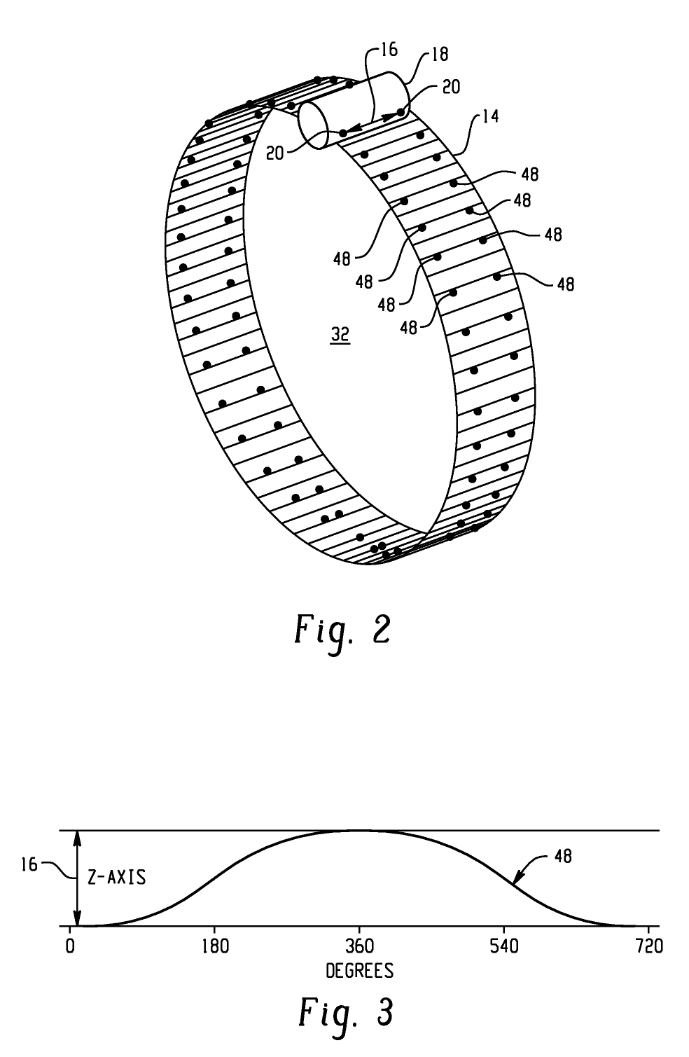

FIG. 2 illustrates an exemplary half-cycle closed helical (HCCH) radiation source orbit over two gantry revolutions.

FIG. 3 illustrates the HCCH orbit along the z-axis as a function of gantry rotation angle in degrees.

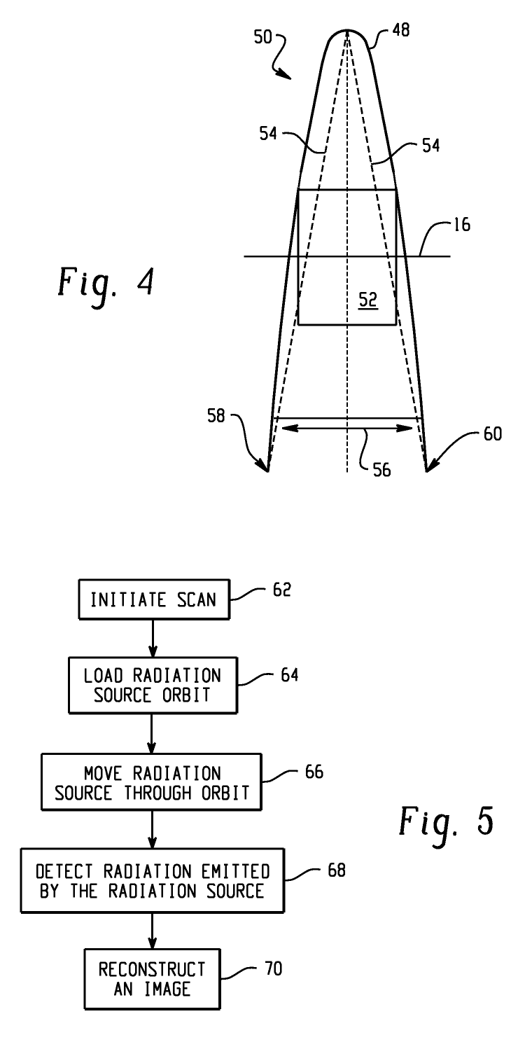

FIG. 4 illustrates an exemplary profile of the HCCH path along the-z axis.

FIG. 5 illustrates a method for scanning a subject using a HCCH radiation source orbit.

With reference to FIG. 1, a medical imaging system 10 is illustrated. The medical imaging system 10 uses various techniques to acquire suitable data of a region or volume interest of a subject and image slices, multi-dimensionally rendered or other of the region or volume of interest therefrom while making efficient use of the detectors. For instance, the medical imaging system 10 can employ one or more different x-ray source orbits, paths, trajectories, etc, around the subject while irradiating the subject and detecting transmission, scatter, etc. radiation. Examples of such orbits include a circular (or axial), a circular/line, a saddle, an ellipse, a helical segment, and/or a half-cycle closed helical, and/or derivations thereof, and/or other orbital paths.

Using the half-cycle closed helical, the medical imaging system 10 can acquire a complete data set, or complete sampling (e.g., in a single non-discontinuous motion), of a volume of interest (VOI) for generating images of the VOI (e.g., via 180 degree reconstruction, etc.). Such acquisition can be achieved while efficiently using the detectors (e.g., minimizing detector width along the z-axis, etc.), reducing the number of gantry revolutions (and thus, scan time and/or patient dose), and/or increasing ease of repeatability.

As depicted, the medical imaging system 10 includes a CT scanner 12. The CT scanner 12 includes a rotating gantry 14, which rotates about a z-axis 16. The rotating gantry 14 supports one or more x-ray tubes 18, which generate at least one radiation beam (e.g., conical, fan, etc.) at one or more positions, such as focal spots, of one or more radiation sources 20. One or more of the focal spots may be dynamic in that they can rapidly shift or deflect to a plurality of positions during rotation of the x-ray tube 18 around the gantry 14.

In one instance, the radiation source 20 is movable along at least the z-axis 16. Such movement can be achieved by physically or mechanically moving the x-ray tube 18 along the z-axis and, hence, sweeping the radiation source 20 along the z-axis 16, and/or electronically by deflecting the x-ray tube 18 electron beam so that it impinges the anode to the x-ray tube 18 at various positions along the z-axis 16. Physical movement of the x-ray tube 18 and radiation source 20 along the z-axis 16 is coordinated with the rotational movement of the gantry 14 to provide a desired radiation source orbit or trajectory. A gantry orbit bank 22 stores the orbital paths for the scanner 12. As depicted, suitable orbits include a half-cycle closed helical (HCCH) orbit 24, which will be described in greater detail below and, optionally, other orbits.

The gantry 14 also supports x-ray sensitive detectors 30 disposed about the gantry 14 to subtend an angular arc opposite the x-ray source 18 to define an imaging region 32 therebetween. As depicted, the detectors 30 are arranged in a third generation configuration. However, other detector arrangements, including fourth generation, stationary source systems, e-beam scanners, and/or other system geometry arrangements, are also contemplated herein. Each of the detectors 30 includes one or more single or multi-slice regions. The detectors 30 detect radiation emitted by the radiation source 20 that traverses the imaging region 32 and generate corresponding output signals indicative of the detected radiation along a plurality of rays.

The CT scanner 12 further includes a subject (or patient) support 34 that supports a subject within the imaging region 32. The support 34 may be stationary or movable along x, y, and/or z-axes. Such movement allows an operator to guide the subject to a suitable location within the imaging region 32 by moving the support 34 or the support 34 in coordination with the gantry 14 (e.g., tilt, z direction, etc) so as to generate a desired scanning trajectory or orbit.

A computing system 36 facilitates operator interaction with and/or control of the scanner 12. The computing system 36 can be a computer such as a workstation, a desktop, a tower, a laptop, or the like. In one instance, the computing system 36 is a separate general-purpose system that executes applications and/or includes hardware, firmware, and/or software for communicating with the scanner 12. In another instance, the computing system 36 is a dedicated console for the scanner 12.

Software applications executed by the computing system 36 allow the operator to configure and/or control operation of the scanner 12. For instance, the operator can interact with the computing system 36 to select scan protocols, initiate, pause and terminate scanning, view images, manipulating volumetric image data, measure various characteristics of the data (e.g., CT number, noise, etc.), etc. The computing system 36 communicates with a controller 38 that controls the scanner 12 based on the scan parameters. Such communication may include conveying computer readable instructions to configure and/or control the scanner 12 for a particular scan protocol. For example, such instructions may include parameters such as x-ray tube voltage and current, radiation source and x-ray tube position, radiation source orbit,, etc.

Data collected by the detectors 30 is conveyed to a reconstruction system 40 that reconstructs the data to generate volumetric data indicative of the scanned region of the subject. The reconstruction system 40 can be a dedicated system for the scanner 12 and/or a separate general-purpose computer. In addition, the reconstruction system 40 may be an integrated and/or distributed system, wherein subsystems (not shown) such as, but not limited to, a convolver, a backprojector, etc. are part of the same system or distributed over separate subsystems or computers.

An image processor 44 processes the volumetric image data generated by the reconstruction system 40. In one instance, the image processor 44 generates images of the scanned anatomy that are displayed, filmed, archived, forwarded to a treating clinician (e.g., emailed, etc.), fused with images from other imaging modalities, further processed (e.g., via measurement and/or visualization utilities and/or a dedicated visualization system), stored within the storage component 42, etc.

FIG. 2 illustrates an exemplary orbit, path, trajectory, etc. for the previously defined HCCH orbit 24 over two gantry revolutions. In FIG. 2, the radiation source 20 of the x-ray tube 18 moves along the z-axis 16 and follows a helical orbit 48 as the gantry 14 rotates around the imaging region 32. The radiation source 20 sweeps along the z-axis 16 in both directions in a continuous motion such that the radiation source 20 returns to its initial position (or closes the helix) after two gantry rotations, or 720 degrees. Thus, the radiation source 20 helically moves through half a cycle of motion with each gantry rotation, or 360 degrees, and closes after two gantry rotations.

The periodicity of the HCCH orbit 48 renders mechanical based radiation source sweep implementations (e.g., via physical movement of the x-ray tube 18) relatively more feasible than with other orbital paths like the saddle orbit since the x-ray tube 18 can be moved at a relatively slower rate. Various hardware and/or software techniques can be used to compensate for acceleration and/or velocity differences of the movement of the x-ray tube 18 along the-axis 16.

FIG. 3 illustrates the HCCH orbital path 48 along the z-axis 16 as a function of gantry rotation angle in degrees over 720 degrees. In FIG. 3, the path 48 is shown as a smooth continuous function (sinusoidal); however, other paths, though not preferred, such as discontinuous, triangular, etc. are also contemplated herein. As described in more detail below, complete sampling of the VOI is achieved with data collected over about one and one quarter revolutions, or about 450 degrees.

FIG. 4 illustrates an exemplary profile 50 of the radiation source 20 following the HCCH path 48 along the-z axis 16 over two gantry revolutions (e.g., starts at 58 or 60 travels 360 degrees to 60 or 58 and then returns over 360 degrees back to 58 or 60). For complete sampling of a cylindrical VOI 52, the radiation source trajectory 48 encloses the VOI 52 as shown. An approximate extent (or z-axis width) of each of the detectors 30 for acquiring the VOI 52 is defined by rays 54 of the x-ray beam and illustrated at 56. The source trajectory 48 makes efficient use of the detector along the z-axis 16, for a 180 degree reconstruction, of substantially all voxels within the VOI 52. By way of non-limiting example, using the HCCH source trajectory 48 for a 120 mm long VOI, the spot sweep is about 226.5 mm with a detector extent of approximately 210 mm. Using a saddle trajectory for the same VOI, the detector extent would increase to about 328 mm.

As the radiation source 20 moves through a cycle and returns to its starting position after two gantry revolutions, radiation is detected by the detectors 30. All or a subset of the 720 degrees worth of detected data is used to reconstruct an image(s). For instance, each voxel can be reconstructed from at least 180 degrees plus fan angle. In order to reconstruct all voxels in the VOI 52, a subset of data collected from about one and one quarter revolutions is used. Thus, when performing a 180 degree reconstruction, the reconstruction system 40 uses a suitable portion of detected data collected over two gantry revolutions to reconstruct images. That is, a desired subset of the data collected over two revolutions may be selected for reconstruction. In another instance, since 720 degrees worth of data is not required for a 180 degree reconstruction, the x-ray tube 18 can be turned off after enough data is collected for reconstruction.

Upon acquiring the data, a voxel-dependent 180 degree reconstruction can be performed to image the VOI 52. After parallel rebinning of the projections, pi-surfaces can be identified that intersect the VOI 52 at a given pair of source angles. Voxels at the intersection are reconstructed using the 180 degree range of views between the pair of source angles. For each 180 degree reconstruction, more than 180 degrees plus fan angle worth of data can be used, if desired, to minimize motion differences at the beginning and the end of each subset of data. For example, overlapped data acquired at different times can be averaged.

FIG. 5 illustrates a non-limiting method for scanning a subject with the medical imaging system 10. At reference numeral 62, an operator interacts with scanner software applications executed by the computing system 36 to configure and/or control operation of the scanner 12 to scan a subject in the imaging region 32. For this example, assume the operator has selected a 180 degree reconstruction technique, either directly and/or indirectly through selecting a scan protocol, etc. that uses 180 degree reconstruction. Also assume that for the selected procedure the radiation source 20 is moved along the z-axis 16 by mechanically moving the x-ray source 18 (e.g., physical movement) and/or electronically sweeping the generated beam through the HCCH orbit 24 (which is stored in the gantry orbit bank 22). As described above, using the HCCH orbit 24 the radiation source 20 sweeps along the z-axis 16 in both directions in a continuous motion such that the radiation source 20 moves through and closes a helix path (or returns to its initial position) after two gantry rotations. The computing system 36 communicates this and other information to the controller 38.

At reference numeral 64, the control system 38 conveys control commands, which include instructions and/or parameters for moving the radiation source 20 through the HCCH orbit 24 to the scanner 12. As described above, radiation source 20 movement is achieved by mechanical and/or electronic techniques. At 66, the scanner 12 operates under the control commands and the radiation source 20 is moved through the HCCH orbit 24 while generating an x-ray beam. At 68, the detectors 30 detect the emitted radiation and produce signals indicative thereof. At 70, the reconstruction component 40 reconstructs the signals, based on the selected 180 degree reconstruction technique, and the image processor 44 processes the reconstructed data to generate corresponding images. As described above, all or a subset of the 720 degrees worth of data is used to reconstruct images. About one and a quarter gantry revolutions worth of data provides a complete set of data for reconstructing the images. The images can be stored in the storage component 42 and/or provided to the computing component 36 for visual observance by the operator, filmed, further processed, etc.

The systems and/or methods described herein and/or derivations thereof can be used with low, mid, and/or high end systems, including applications for partial and/or whole organ imaging such as the heart, perfusion imaging of the heart, brain, etc., as well as other applications.

The invention has been described with reference to the preferred embodiments. Of course, modifications and alterations will occur to others upon reading and understanding the preceding description. It is intended that the invention be construed as including all such modifications and alterations insofar as they come within the scope of the appended claims.

1. A tomographic apparatus comprising:
a radiation source that sweeps along a z-axis and returns to its initial position in coordination with about two revolutions of the radiation source around an imaging region with a frequency of about half a frequency of a revolution of the radiation source around the imaging region and;
at least one radiation sensitive detector that detects radiation emitted from the radiation source that traverses a volume of interest within the imaging region and generates data indicative of the detected radiation; and
a reconstruction system that reconstructs the detected data to generate an image of the volume of interest.

2. The apparatus of claim 1 wherein the detected radiation represents a complete sampling of the volume of interest.

3. The apparatus of claim 1 wherein the reconstruction system reconstructs the VOI using data collected over at least one and one quarter revolutions of the radiation source about the imaging region.

4. The apparatus of claim 1 further including a gantry orbit bank that stores a half-cycled closed helical orbit wherein the radiation source follows the half-cycled closed helical orbit as the radiation source rotates about the imaging region.

5. The apparatus of claim 1 wherein the reconstruction system reconstructs the data using a 180 degree reconstruction technique.

6. The apparatus claim 1 wherein the radiation source sweeps along the z-axis by physically moving an x-ray source producing the radiation source in the z-axis.

7. The apparatus of claim 1 wherein the radiation source electronically sweeps along the z-axis.

8. The apparatus of claim 1 wherein the radiation source sweeps along the z-axis in a continuous motion and forms a closed path.

9. The apparatus of claim 1 wherein the at least one sensitive radiation detector acquires data to reconstruct images for at least one of cardiac and perfusion scanning.

10. A computed tomography reconstruction method comprising:
sweeping a radiation source along a z-axis in a closed helical path in which the radiation source returns to its initial starting position with a frequency of about half a frequency of a beam emitted by the radiation source that rotates about an imaging region;
detecting radiation emitted from the radiation source that traverses the imaging region;
generating data indicative of the detected radiation; and
reconstructing an image of a subject within the imaging region from the data.

11. The method of claim 10 wherein sweeping the radiation source and rotating the beam are coordinated over two revolutions of the beam around the imaging region.

12. The method of claim 10 wherein the detected radiation represents a complete data set of a volume of interest of the subject in the imaging region.

13. The method of claim 10 further including using a subset of the data to reconstruct the image.

14. The method of claim 10 further including reconstructing data corresponding to one and one quarter rotations of the radiation source about the imaging region to generate the image.

15. The method of claim 10 further including sweeping the radiation source through a half-cycle closed helical orbit.

16. The method of claim 10 further including employing a 180 degree reconstruction technique to reconstruct the image.

17. The method of claim 10 further including physically moving an x-ray source along the z-axis to sweep the radiation source.

18. The method of claim 10 further including electronically sweeping the radiation source.

19. The method of claim 10 further including detecting radiation used to reconstruct images for at least one of cardiac and perfusion scanning.

20. An apparatus comprising:
means for sweeping a radiation source along a z-axis with a frequency of about half a frequency of a revolution of the radiation source around an imaging region;
means for detecting radiation emitted from the radiation source over at least one and a quarter revolutions and generating data indicative of the detected radiation; and
means for reconstructing each voxel within a volume of interest residing within the imaging region with the data.
